(19) United States
(12) Patent Application Publication
(10) Pub. No.: US 2012/0320568 A1
(43) Pub. Date: Dec. 20, 2012

(54) MEDICAL/DENTAL HEADLIGHT SYSTEM WITH IMPROVED COLOR RENDITION

(75) Inventor: Byung J. Chang, Ann Arbor, MI (US)

(73) Assignee: General Scientific Corporation, Ann Arbor, MI (US)

(21) Appl. No.: 13/162,918

(22) Filed: Jun. 17, 2011

(51) Int. Cl. A61B 1/06; F21V 21/084

(52) U.S. Cl. 362/105

(57) ABSTRACT

A head-mounted light source particularly suited to medical and dental applications includes a plurality of light-emitting diodes (LEDs). The LEDs include at least one cool white LED; at least one warm white LED; and at least one red LED. By combining a cool white, warm white and red LED, a more natural spectrum is produced. In one preferred embodiment, the system includes a single cool white LED, a single warm white LED, and a single red LED. In an alternative embodiment, the system includes 3 warm white LEDs, 2 cool white LEDs and 2 red LEDs which may be arranged in a hexagonal close-packed pattern.

### FIELD OF THE INVENTION

This invention relates generally to head-mounted lights of the type used by medical and dental professionals and, in particular, to a headlamp system that uses multiple light-emitting diodes to improve the color characteristics of the beam.

### BACKGROUND OF THE INVENTION

Many medical and dental professionals use head-mounted lights to better visualize procedures. However, visible light-emitting diode (LED) lights have limited bandwidths. For example, cool LED lights produce a strong blue spectrum and weak red spectrum.

FIG. 4 shows the light generated by a typical cool white LED. As evident from this diagram, the light produced by a typical (phosphor-based) white LED has a dominant peak around 450 and a lower peak from around 500-600 nm. Such a spectrum can produce eye strain for many professionals, particularly during prolonged medical and dental procedures.

### SUMMARY OF THE INVENTION

This invention resides in a headlamp assembly particularly suited to medical and dental professionals. The assembly includes a housing with a mechanism enabling the housing to be coupled to a user's head and a plurality of light-emitting diodes (LEDs) in the housing. The LEDs include at least one cool white LED; at least one warm white LED; and at least one red LED. By combining a cool white, warm white and red LED, a more natural spectrum is produced. In one preferred embodiment, the system includes a single cool white LED, a single warm white LED, and a single red LED. In an alternative embodiment, the system includes 3 warm white LEDs, 2 cool white LEDs and 2 red LEDs which may be arranged in a hexagonal close-packed pattern.

### BRIEF DESCRIPTION OF THE FIGURES

FIG. 1 is a drawing that shows a medical/dental light using a single white LED and beam-forming optics;

FIG. 2 illustrates an embodiment of the invention using three different LEDs to produce a more natural spectrum;

FIG. 3 illustrates an embodiment of the invention using an arrangement of seven LEDs;

FIG. 4 shows the light produced by a typical cool white LED;

FIG. 5 shows the light produced by a typical warm white LED;

FIG. 6 shows the light produced by a typical red LED; and

FIG. 7 shows the light produced by the invention.

### DETAILED DESCRIPTION OF THE INVENTION

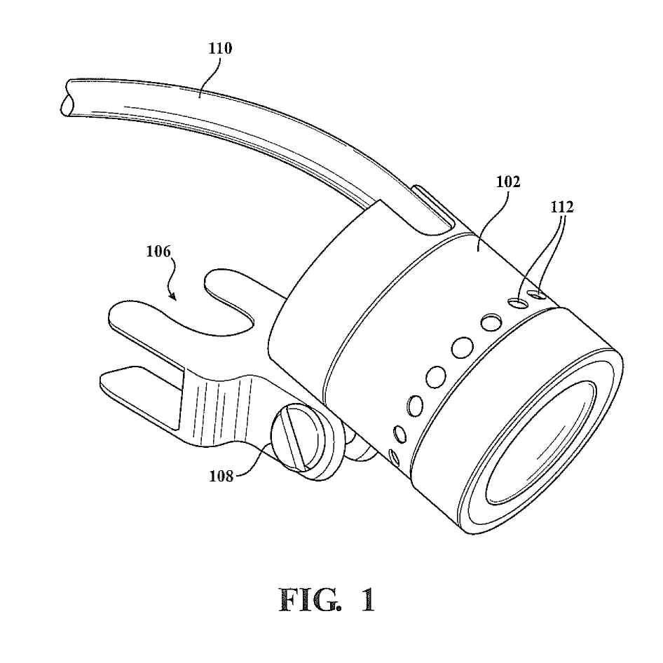

By way of background, FIG. 1 illustrates a typical LED headlamp of the type used by medical and dental professionals. This system includes a housing 102 including one or more beam-forming lenses. The housing 102 may be coupled to a clip-on unit 106 through a swivel 108 enabling the assembly to be removable mounted to optical loupes of the type offered by Surgitel Products of Ann Arbor, Mich. A light-emitting diode (LED) is also contained in the housing 102, with electrical cord 110 providing electrical power from a remote power supply such as a battery pack (not shown). Alternatively, cord 110 may be a fiber bundle providing light from a remote light source. The housing 102 may be made of aluminum or other metal, with holes 112 being used for more efficient cooling. While the inventions described herein may be used with the LED headlamp just described, the invention is not limited to this exact configuration, and may take advantage of any style of housing, alternative head-mounted attachment structures, and beam-forming optics.

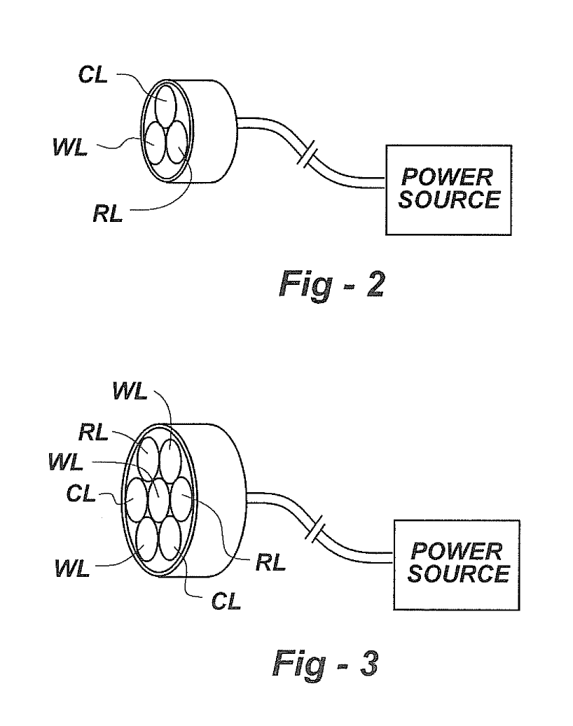

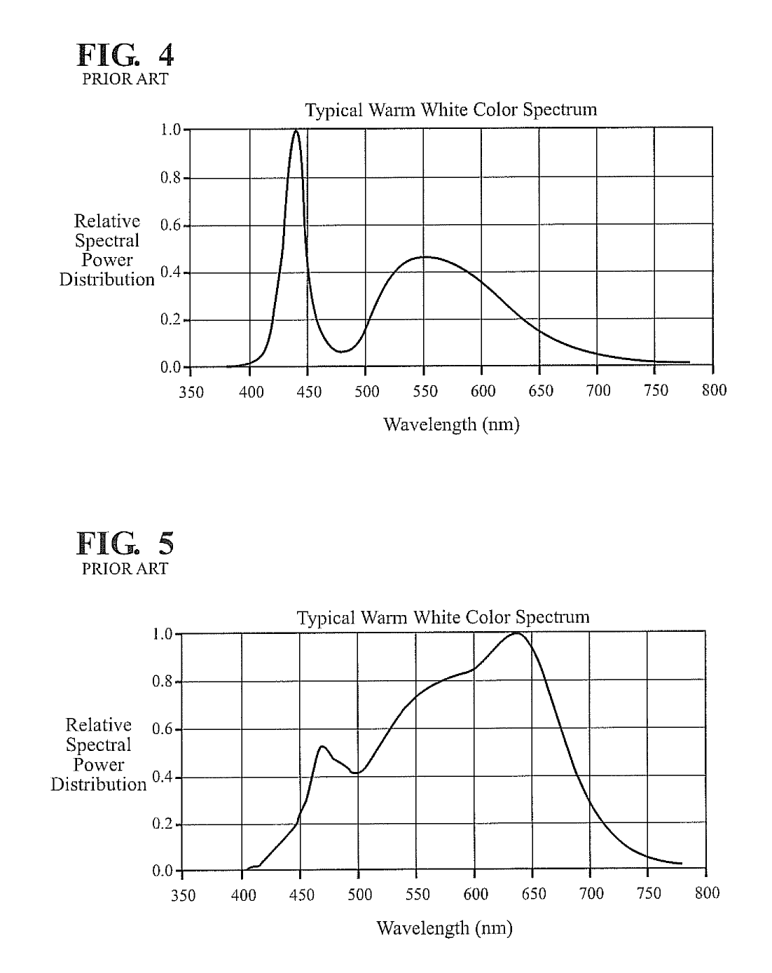

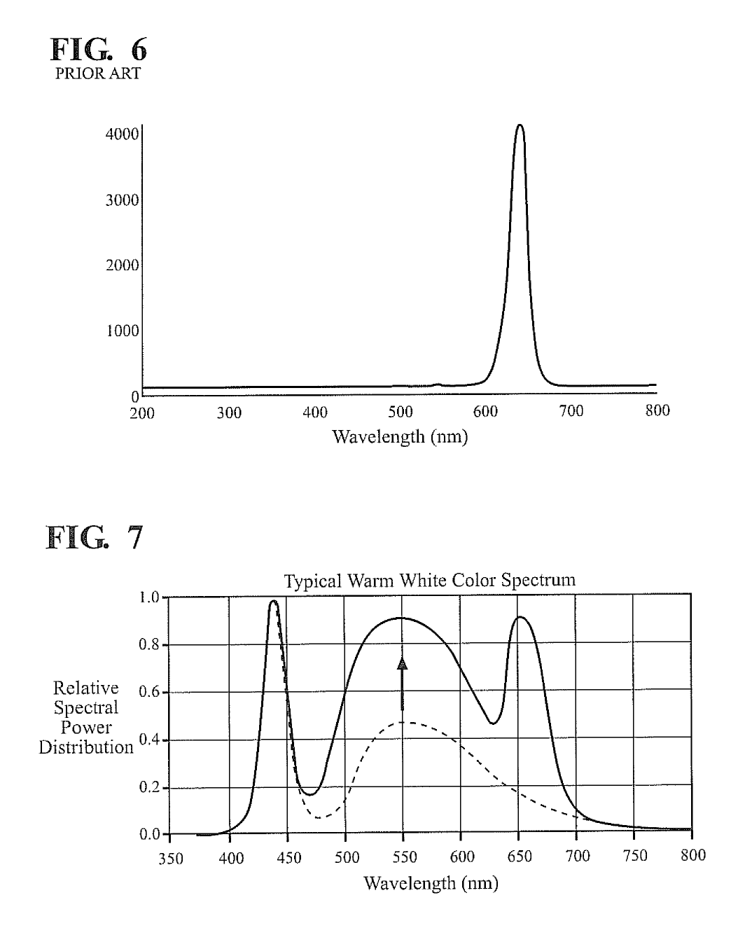

FIG. 2 illustrates an embodiment of the invention using three different LEDs to produce a more natural spectrum. This embodiment uses one at least one cool white LED, at least one warm white LED, and at least one red LED. (Warm white LEDs are typically made with multiple LEDs as opposed to a single blue emitter and phosphor combination). FIG. 4 shows the light produced by a typical cool white LED FIG. 5 shows the light produced by a typical warm white LED, and FIG. 6 shows the light produced by a typical red LED. By combining a cool white, warm white and red LED, a more natural spectrum is produced, as shown in FIG. 7.

The invention is not limited to a 3-LED combination. FIG. 3 shows a system constructed in accordance with the invention that uses seven (7) emitters, including three (3) warm white emitters, 2 cool white and 2 red emitters. The spectrum produced is similar to the shown in FIG. 7, wherein the peak between 500 and 600 nm produced by the cool white emitters is essentially pushed up to generate a more even spectral distribution. In the embodiment of FIG. 3, the 7 emitters are preferably hexagonally close-packed to save space.

I claim:

1. A headlamp assembly particularly suited to medical and dental professionals, comprising:
a housing including a mechanism enabling the housing to be coupled to a user's head; and
a plurality of light-emitting diodes (LEDs) in the housing, including:
at least one cool white LED;
at least one warm white LED; and
at least one red LED.

2. The headlamp assembly of claim 1, including a single cool white LED, a single warm white LED, and a single red LED.

3. The headlamp assembly of claim 1, including 3 warm white LEDs, 2 cool white LEDs and 2 red LEDs.

4. The headlamp assembly of claim 1, including:
3 warm white LEDs, 2 cool white LEDs and 2 red LEDs; and
wherein the LEDs are arranged in a hexagonal close-packed pattern.
